(19) United States
(12) Patent Application Publication
(10) Pub. No.: US 2022/0170118 A1
(43) Pub. Date: Jun. 2, 2022

(54) METHODS AND SYSTEMS FOR DETERMINING VIRUSES IN BIOLOGICAL SAMPLES USING A SINGLE ROUND BASED POOLING

(71) Applicants: Indian Institute of Technology Bombay, Mumbai (IN); Institute for Stem Cell Science and Regenerative Medicine, Bangalore (IN); National Centre for Biological Sciences, Bangalore (IN); Algorithmic Biologics Private Limited, Bangalore (IN)

(72) Inventors: Manoj Gopalkrishnan, Mumbai (IN); Sabyasachi Ghosh, Mumbai (IN); Ajit .V Rajwade, Mumbai (IN); Dasaradhi Palakodeti, Bangalore (IN); Sandeep Krishna, Bangalore (IN)

(21) Appl. No.: 17/535,998

(22) Filed: Nov. 27, 2021

(30) Foreign Application Priority Data
Nov. 27, 2020 (IN) 202021051801

(51) Int. Cl. C12Q 1/70; G16B 25/20; G16H 10/40

(52) U.S. Cl. CPC C12Q 1/70; G16H 10/40; G16B 25/20

(57) ABSTRACT

Methods and systems for determining viruses in biological samples using a single round based pooling. Embodiments disclosed herein relate to quantitative testing of biological samples, and more particularly to a quantitative, non-adaptive and single round pooling method for testing of viruses (for example: Coronavirus disease of 2019 (COVID-19), Severe Acute Respiratory Syndrome (SARS), or the like) in biological samples of individuals.

### CROSS-REFERENCE TO RELATED APPLICATIONS

This application claims priority to Indian patent application no. 202021051801 filed on Nov. 27, 2020, the complete disclosure of which, in its entirety, is herein incorporated by reference.

### TECHNICAL FIELD

Embodiments disclosed herein relate to quantitative testing of biological samples, and more particularly to a quantitative, non-adaptive and single round pooling method for testing of viruses (for example: Coronavirus disease of 2019 (COVID-19),

Severe Acute Respiratory Syndrome (SARS), or the like) in biological samples of individuals.

### BACKGROUND

One or more diseases related to single-stranded ribonucleic acid (RNA) viruses (for example: Coronavirus disease of 2019 (COVID-19), Severe Acute Respiratory Syndrome (SARS), or the like)) have led to widespread lockdowns in several countries and impacted economy of the several countries. Early identification of infected individuals may enable quarantining of such individuals, thereby controlling spread of the disease. A widespread testing of the virus has to be performed for the early identification of the infected individuals. However, the widespread testing may not be an available option in many countries due to constraints on resources such as time, basic equipment, skilled manpower, reagents, and so on.

In conventional approaches, for the widespread testing of the virus, pooling strategies may be used. In accordance with the pooling strategies, the biological samples of the individuals have been combined into pools and tested together using a quantitative reverse transcription Polymerase Chain Reaction (PCR) (RT-qPCR) method. If a pool is tested negative, the biological samples of all the individuals within the pool may be determined as free of virus. If the pool is tested positive, then the biological samples of one or more individuals within the pool may include the virus. Further, the biological samples of the individuals in such a pool may be tested individually in a second round to determine the samples of the individuals that include the virus (i.e., the infected individuals), thereby augmenting testing capabilities. However, such approaches may not substantially improve throughput, since two rounds of testing may be required to identify positive samples (that are the samples including the virus)/infected individuals.

Further, a challenge that such a pooling strategy has to confront is whether to first perform pooling of the biological samples of the individuals and then perform a RNA extraction on the pools (to extract the RNA from the biological samples) or whether to individually perform the RNA extraction on each of the biological samples of the individuals and then subsequently perform the pooling of the RNA. If the RNA extraction is performed individually for each biological sample, then a positive PCR control has to be performed on a RP gene of each and every sample, that may nullify any gains from the pooling. If the RNA extraction is performed for each pool, then the second round of testing requires a new round of RNA extraction for all the biological samples of the individuals, which may slow down the process of testing the virus in the biological samples.

### OBJECTS

The principal object of embodiments herein is to disclose a quantitative, non-adaptive and single round pooling method for determining viruses (for example: Coronavirus disease of 2019 (COVID-19), Severe Acute Respiratory Syndrome (SARS), or the like) in biological samples of individuals.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, throughout which like reference letters indicate corresponding parts in the various figures. The embodiments herein will be better understood from the following description with reference to the drawings, in which:

FIG. 1 depicts a system for determining viruses in biological samples of individuals, according to embodiments as disclosed herein;

FIG. 2 is an example block diagram depicting various components of a sample decoding device for determining the viruses in the biological samples of the individuals, according to embodiments as disclosed herein;

FIG. 3 depicts a test and status recovering manager performable in the sample decoding device, according to embodiments as disclosed herein;

FIGS. 4a, 4b and 4c are example conceptual diagrams depicting determination of the viruses in the biological samples of the individuals using the quantitative, non-adaptive and single round pooling method, according to embodiments as disclosed herein;

FIG. 5 is an example diagram depicting recovering status of a plurality of biological samples using the quantitative, non-adaptive and single round pooling method, according to embodiments as disclosed herein;

FIGS. 6a. 6b, .6c, and 6d are example diagrams depicting testing of the biological samples of 40 patients using the quantitative, non-adaptive and single round pooling method, according to embodiments as disclosed herein;

FIGS. 7a. 7b, 7c, and 7d are example diagrams depicting testing of the biological samples of 60 patients using the quantitative, non-adaptive and single round pooling method, according to embodiments as disclosed herein;

FIG. 8 is an example table depicting an improved throughput in recovering the status of the biological samples using the quantitative, non-adaptive and single round pooling method compared to a conventional approach, according to embodiments as disclosed herein; and

FIG. 9 is a flow diagram depicting a method for determining the viruses in the biological samples of the individuals, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Embodiments herein disclose methods and systems for determining viruses (for example: Coronavirus disease of 2019 (COVID-19), Severe Acute Respiratory Syndrome (SARS), or the like) in biological samples of individuals using a quantitative, non-adaptive and single round pooling.

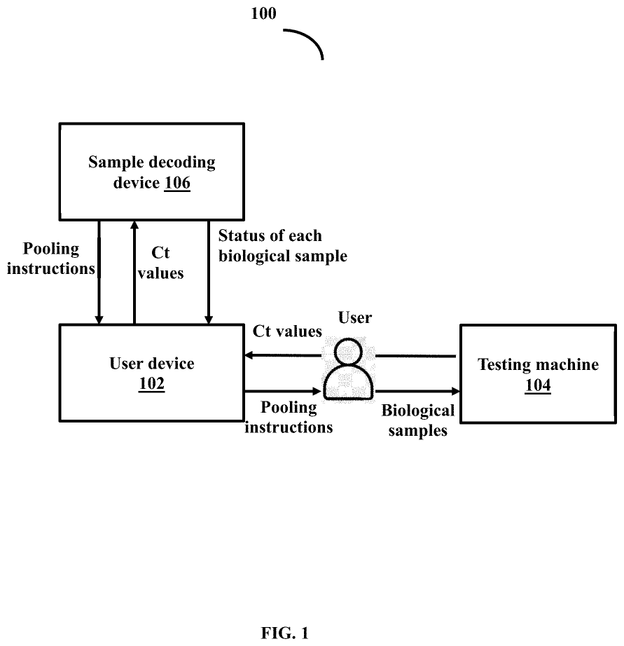

Referring now to the drawings, and more particularly to FIGS. 1 through 9, where similar reference characters denote corresponding features consistently throughout the figures, there are shown embodiments.

FIG. 1 depicts a system 100 for determining viruses in biological samples of individuals, according to embodiments as disclosed herein. The system 100 referred herein may be configured to determine the viruses present in biological samples of individuals/humans. In an embodiment, the viruses may be single-stranded Ribonucleic acid (RNA) viruses. Examples of the viruses may be, but is not limited to, coronavirus disease 2019 (COVID-19), Severe Acute Respiratory Syndrome (SARS), Poliovirus, Rhinovirus, Hepatitis A virus or any other virus that may be tested based on extraction of RNA from the biological samples. In an embodiment, the biological samples may include at least one of, but is not limited to, a fluid sample, a saliva sample, a pulmonary lave, a blood sample, a skin sample, a bone or any other tissue biopsy or sample, or any other sample from which RNA may be derived. Examples of the fluid sample may be, but is not limited to, nasopharyngeal swab (that may be collected from a nose of the individual), anterior nares swab (that may be collected from the nose of the individual), oropharyngeal swab (that may be collected from a throat of the individual), and so on.

In an embodiment, the system 100 may determine the viruses in the biological samples of the individual in a single round using a quantitative, and non-adaptive pooling method. Determining the viruses in the biological samples involves:
determining a pooling matrix for the biological samples to be tested, based on at least one of sparse expanders, explicit optimization of a loss function in a gradient descent and simulated annealing fashion, and Steiner triples, wherein the pooling matrix indicates a total number of pools to be created for testing the biological samples and two or more pools for including each biological sample;
including/pipetting each of the biological samples into the two or more pools, that have been indicated in the pooling matrix;
performing a quantitative reverse transcription Polymerase Chain Reaction (PCR) (RT-qPCR) test on each pool of the biological samples to obtain input cycle threshold values (Ct values) of each pool, wherein the Ct values of each pool are converted into a quantitative measure of viral load of each pool;
outputting a result of each biological sample in the single round, based on the quantitative measure of viral load of each pool and a compressed sensing method, wherein the result of the biological sample indicates whether the biological sample includes the viruses or not.

In an embodiment, the system 100 may also use the pooling method to perform serological antibody tests for determining presence of virus antibodies in at least one of, but not limited to, plasma, blood samples, or the like of the individual. In an embodiment, the system 100 may also use the pooling method for performing quantitative tests (for example, determining level of potassium in the biological samples, or the like).

The system 100 includes user devices 102, testing machines 104, and a sample decoding device 106.

The user device(s) 102 referred to herein may be a device used by a user. The user may be at least one of, but not limited to, a trained technician, a lab operator, or any other person who performs the RT-qPCR tests on the biological samples of the individuals using the testing machine 104. The user device 102 may be configured with an application provided by the sample decoding device 106, that aids the user in performing the RT-qPCR tests on the biological samples of the individuals. The user device 102 configured with the application, receives a sequence of instructions from the sample decoding device 106 and provides the received sequence of instructions to the user to aid and direct the user in pipetting/combining the biological samples of the individuals into the appropriate pools for performing the RT-qPCR tests on each pool. The user device 102 also receives the input cycle threshold values of each pool of biological samples from the user, on competing the RT-qPCR tests on each pool and forwards the received input cycle threshold values of each pool of biological samples to the sample decoding device 106 for determining the results/status of each of the biological samples. The results/status of each biological sample may indicate whether the viruses are present in the biological sample or not. The user device 102 also receives information about the results of each of the biological samples from the sample decoding device 106 and provides the received information about the results to the user. The user device 102 may provide the instructions and the information about the results to the user in a form of at least one of, text, visual cues/alerts, audio, graphics, and so on.

The testing machine 104 may include at least one of, RNA extraction kits, PCR reaction plates, and so on. The RNA extraction kit may be used by the user to extract the RNA from the collected biological samples of the individuals. The PCR reaction plate may be a flat plate including a plurality of wells. The user may pipette the RNA extracted from the biological samples of the individuals into the wells of the PCR reaction plate. Alternatively, the user may use liquid handling robots to pipette the RNA extracted from the biological samples of the individuals into the wells of the PCR reaction plate.

The sample decoding device 106 referred to herein may be at least one of a cloud computing device (can be a part of a public cloud or a private cloud), a server, a computing device, and so on. The server may be at least one of a standalone server, a server on a cloud, or the like. The computing device may be, but is not limited to, a personal computer, a notebook, a tablet, desktop computer, a laptop, a handheld device, a mobile device, and so on. Also, the sample decoding device 106 may be at least one of, a microcontroller, a processor, a System on Chip (SoC), an integrated chip (IC), a microprocessor based programmable consumer electronic device, and so on. The sample decoding device 106 may be connected with the user devices 102 using a communication network. Examples of the communication network may be, but are not limited to, the Internet, a wired network, a wireless network (a Wi-Fi network, a cellular network, a Wi-Fi Hotspot, Bluetooth, Zigbee and so on) and so on.

The sample decoding device 106 may be configured to provide instructions to the user for pooling and testing the biological samples, on receiving a request from the user. The request may include information about at least one of, a name of a test (with date and time), a size of the test, and so on. The size of the test may indicate a number of biological samples to be tested and a number of positives estimated (by the user) out of the total number of biological samples to be tested. The number of positives estimated may correspond to the biological samples (from the number of biological samples to be tested) that include the viruses. In an example, the user may estimate the number of positives based on past experience of testing the biological samples. For example, the number of positives estimated out of 40 samples may be 1, 2, 3, 4, or the like. The instructions may include information about at least one of, a total number of pools/tests for the number of biological samples to be tested, two or more pools for each biological sample (i.e., the two or more pools in which each biological sample may be pipetted/included), or the like.

For providing the instructions to the user, the sample decoding device 106 creates a pooling/sensing matrix for the requested size of test. The pooling matrix includes a plurality of rows and columns The plurality of columns indicates the number of biological samples to be tested and the plurality of rows indicates the number of tests/pools to be created for testing of the biological samples. The matrix may include values/entries of 0 and 1. The values 1 with respect to each column indicates the pools for including the biological sample corresponding to each column In an embodiment, each biological sample may be included in three pools for testing. Thus, each column of the matrix may include only three values of 1's representing three pools for the biological sample corresponding to each column.

For example, consider that the sample decoding device 106 receives the request from the user for testing of 40 biological samples. In such a scenario, the sample decoding device 106 creates the pooling matrix, wherein the pooling matrix includes 16 rows and 40 columns. The 40 columns indicate the 40 biological samples to be tested and the 16 rows indicate that the 16 pools have to be created for testing of the 40 biological samples. The values/entries of 1's of each column indicates the pools for including the respective biological sample.

In an embodiment, the matrix may be created by generating a function that is a dot product of pair of columns and optimizing the function in a gradient descent and a simulated annealing fashion. In an example, the created pooling matrix A may be represented as:

$$A = {( {A}_{j i} )}_{m \times n}$$

wherein the pools may be numbered as 1, 2, 3 . . . n and indexed by and the biological samples may be numbered as 1, 2, 3 . . . n and indexed by ‘i’. The entries of the pooling matrix A may be binary/non-negative entries.

The pooling matrix A may be an adjacency matrix of expander graphs/sparse expanders. A left-regular bipartite graph G((V₁, V₀), ∈⊆V₁×V₀ with degree of each vertex in V₁ being d, may be referred as (k, 1−∝) expander graph for integer k>0 and real value α∈(0,1), if for every subset S⊆V₁ with |S|≤k. Further, a union set of neighbors of all nodes of S may be represented as |N(S)|, wherein |N(S)|≥(1−α) d|S|. Intuitively, the bipartite graph may be the expander graph, if every larger subset compared to other subsets has a large boundary. Further, the pooling matrix with (k, 1−∝) expander graph obeys Restricted Isometry Property -1 (RIP-1) (a version of RIP, with l₁ norm). For example, for any k-sparse vector x, the following relationship holds if the pooling matrix A obeys the RIP-1 property of order k:

$$( 1 - 2 ∝ ) d {‖ x ‖}_{1} \leq {‖ Ax ‖}_{1} \leq d {‖ x ‖}_{1}$$

Further, the sample decoding device 106 may perform a gradient descent method or the stimulated annealing method to update the entries/values of the pooling matrix A, starting with an initial condition.

In another embodiment, the sampling decoding device 106 creates the pooling matrix using a Steiner triple system. The pooling matrix created using the Steiner triple system indicates the number of pools for testing of the biological samples and three pools for including each biological sample. The Steiner triple system may consist of ‘n’ number of rows and ‘n(n−1)/6’ number of columns, with entries 0 or 1, such that each column has exactly three 1's and no two columns have the dot product more than 1. Each column/biological sample may be the Stiner Triples and each row/pool may be a n-element set S. As the pooling matrix created using the Steiner triples systems may be the sparse matrix, since each column has only the three 1's. The dot product between the two columns being at most 1 ensures that no two samples participate in more than one test/pool together, which has favorable consequences in terms of defining an upper bound on a mutual coherence of the pooling matrix. In an example, the upper bound on the mutual coherence of the pooling matrix may be defined as:

$$μ ( A ) = {max}_{i \neq j} \frac{\vert {A}_{i}^{t} {A}_{j} \vert}{{‖ {A}_{i} ‖}_{2} {‖ {A}_{j} ‖}_{2}}$$

wherein, μ(A) indicates the mutual coherence and Aᵢ indicates i^{th} column of the pooling matrix A.

On creating the pooling matrix, the sample decoding device 106 determines the number of pools to be created for the biological samples using the rows of the pooling matrix and the two or more pools for including each biological sample using the entries of each column of the pooling matrix. The sample decoding device 106 provides information about the number of pools to be created for the biological samples and two or more pools determined for each biological sample, as the instructions to the user for pooling and testing the biological samples.

On receiving the instructions from the sample decoding device 106, the user selects the PCR reaction plate with the plurality of wells for testing the biological samples. In an embodiment herein, the wells may correspond to the number of pools.

The user selects the PCR reaction plate according to the number of pools/tests indicated in the received instructions. The user performs numbering of the biological samples and the wells of the PCR reaction plate in a matrix format, according to the instructions received from the sample decoding device 106. The user then performs the pooling of the biological samples by pipetting each of the biological samples into the different numbered wells/pools of the PCR reaction plate, according to the instructions received from the sample decoding device 106. In an embodiment, pooling of the biological samples may involve extracting/isolating (using suitable RNA extraction kits) the RNA fragments from each of the biological sample and then subsequently pipetting the extracted RNA fragment into the two or more wells/pools of the PCR reaction plate, according to the instructions received from the sample decoding device 106. In another embodiment, pooling of the biological samples may involve pipetting the extracted RNA fragment into the two or more wells/pools of the PCR reaction plate, according to the instructions received from the sample decoding device 106 and extracting/isolating the RNA fragments from the biological samples of each pool.

In an example herein, the user may use the extracted RNA fragments corresponding to the biological samples of each pool of length 1 kb (stock concentration 10 ng/μL) may be used as a proxy for preliminary testing The user may dilute the RNA fragments to clinically relevant concentrations of 0.05 pg/μL (˜10⁵ copies/μL), 5 fg/μL (˜10⁴ copies/μL) and 0.5 fg/μL (˜10³ copies//μL) by serial dilution from the stock. In another example, the user may use a circular DNA plasmid containing the complete nucleocapsid gene from the SARS-CoV-2 virus (Integrated DNA Technologies, 2019-nCoV_N_Positive Control, Catalog #10006625) for the testing. The user may dilute the

DNA plasmid to clinically relevant concentrations of 104 copies/μL and 103 copies/μL.

On diluting the RNA fragments/DNA plasmid (that have been) pipetted in the wells of the PCR reaction plate, the user may perform the RT-qPCR test on the biological samples (i.e., the combined samples) of each pool/well of the PCR reaction plate using a PCR machine. The RT-qPCR test may involve the qPCR amplification from the RNA templates/fragments or the qPCR amplification from the DNA templates.

The qPCR amplification from the RNA templates involves synthesizing the cDNA from the RNA templates using First Strand Invitrogen SuperScript II system (Invitrogen, catalog #18064-014). A mix of RNA and a genespecific reverse transcription primer may be denatured at 95° C. for 5 minutes and annealed at 50° C. for 5 minutes prior to the addition of the other reaction components. After adding the other components, the reverse transcription reaction may be carried out for 1 hour at 50° C. and followed by a heat inactivation step for 15 minutes at 85° C. For cDNA templates, qPCR may be performed using the ThermoFisher SYBR GREEN MasterMix system (Thermo Scientific, catalog #K0223). A 35 μl qPCR reaction may be set up using cDNA template (3.5 μL) and forward and reverse primers (2.5 μM each). Each 35 μl reaction may be distributed equally as 3 technical replicates of 10 μl in an optical 384 well plate as technical replicates. The thermocycling conditions may be set as follows: 95° C. denaturation step for 3 minutes followed by 40 cycles of 95° C. for 10 seconds, 55° C. for 15 seconds and 72° C. for 30 seconds. The specificity of the initial test reactions may be verified by primer melt curve analysis and analysis of the amplicons using agarose gel electrophoresis.

The qPCR amplification from the DNA templates involves amplifying the DNA templates (synthesized from the RNA fragments) with TaqPath 1-Step RTqPCR Master Mix, CG (ThermoFisher; catalog #A15299) using the U.S. CDC designed N1 primer and Taqman probe set (IDT; 2019-nCoV RUO Kit, catalog #10006713), by skipping a RT step. In an example, the thermocycling conditions may as follows: 95° C. denaturation step for 2 minutes followed by 45 cycles of 95° C. for 3 seconds, 55° C. for 30 seconds and 72° C. for 30 seconds.

The RT-qPCR test may be intuitively inferred by one of ordinary skill in the art based on its name, and thus, its detailed description is omitted herein.

On performing the RT-qPCR test on each pool, the PCR machine provides amplification curves corresponding to each pool. The amplification curves represent fluorescence intensity (report on a total amount of amplified DNA of the appropriate sequence) against qPCR cycles. The PCR machine may derive the Ct values from the amplification curves for each pool. A smaller Ct value indicates a greater number of copies of the viruses. Deriving of the Ct values from the amplification curves obtained by the RT-qPCR test may be intuitively inferred by one of ordinary skill in the art based on its name, and thus, its detailed description is omitted herein. The PCR machine may derive zero Ct values for the pool, if the pool is negative (i.e., one or more biological samples included the corresponding pool do not include the viruses). The PCR machine may derive the Ct values for the pool, only if the pool is positive (i.e., one or more biological samples in the corresponding pool include the viruses). The PCR machine provides the Ct values of each pool to the user. The user communicates Ct value data to the sample decoding device 106 for recovering/determining the status of each biological sample. The Ct value data may indicate the Ct values of the pool, if the pool is positive. The Ct value data may indicate that the Ct values have not been reached for the pool, if the pool is negative.

The sample decoding device 106 may also be configured to receive the Ct values data of each pool of biological samples and decode the Ct value data of each pool to recover the results/status of the biological samples of the individuals.

For recovering the status/results of each biological sample, the sampling decoding device 106 identify the pools that are negative (i.e., the pools for which the Ct values have not been reached) and the pools that are positive (i.e., the pools for which the Ct values have been determined) based on the Ct values data of each pool. In an embodiment, the sampling decoding device 106 performs a Combinatorial Orthogonal

Matching Pursuit (COMP), a Boolean nonadaptive group testing method on the Ct values data received from the user for the tested pools to identify the pools that are negative and the pools that are positive.

The sample decoding device 106 uses the pools that are identified as positives to recover the status of each biological sample. In an embodiment, the sample decoding device 106 uses a compressed sensing method to recover the status of each biological sample from the pools that are positive. In accordance with the compressed sensing method, the sample decoding device 106 construct a noisy linear equation based on the pooling matrix created for testing of the biological samples and the quantitative measure of the viral load associated with each pool. The sample decoding device 106 solves the noisy linear equation for each of the biological samples to recover the status of each biological sample. In an embodiment, the sample decoding device 106 uses at least one of, a Non-negative least absolute shrinkage and selection operator (NN-LASSO) method, a Non-negative Orthogonal Matching Pursuit (NNOMP) method, a Sparse Bayesian Learning (SBL) method, a brute force search method, or the like to solve the noisy linear equation. The COMP method, the compressed sensing method, the NN-LASSO method, the NNOMP method, the SBL method, the brute force search method may be intuitively inferred by one of ordinary skill in the art based on its name, and thus, its detailed description is omitted herein.

Consider an example scenario, wherein the biological samples that have been tested may be numbered as 1, 2, 3 . . . n and indexed by ‘i’, and the pools/tests created for the biological samples may be numbered as 1, 2, 3 . . . n and indexed by ‘j’. In such a scenario, the sample decoding device 108 identifies the pooling matrix constructed for testing of the biological samples as:

$$A = {( {A}_{j i} )}_{m \times n}$$

wherein, Aⱼᵢ=0 indicates that the i^{th} biological sample is not present in the j^{th} pool, Aⱼᵢ=1 indicates that the i^{th} biological sample is present in the j^{th} pool.

The sample decoding device 106 uses the compressed sensing method and constructs the noisy linear equation by considering that the viral load of i^{th} biological samples as xᵢ and the quantitative measure of the viral load of the j^{th} pool/test as yⱼ. In an example herein, the noisy linear equation may be represented as:

$${\sum }_{i} {a}_{j i} {x}_{i} = {y}_{j} {e}^{∈} j ( noisy linear equation )$$

wherein, ‘∈ⱼ’ may be a mean-zero Gaussian random variable denoting noise in measurement of the Ct values of j^{th} pool.

The sample decoding device 106 solves the noisy linear equation using at least one of, the NN-LASSO method, the NNOMP method, the SBL method, the brute force search method, or the like to determine the value of xᵢ. The value of the xᵢ represents the status/results of the i^{th} biological sample.

The sample decoding device 106 communicates the status/results of the biological samples of the user. Thus, the biological samples may be tested in a single round of testing without a need for a second confirmatory round.

FIG. 1 shows exemplary blocks of the system 100, but it is to be understood that other embodiments are not limited thereon. In other embodiments, the system 100 may include less or a greater number of blocks. Further, the labels or names of the blocks are used only for illustrative purpose and do not limit the scope of the embodiments herein. One or more blocks can be combined together to perform the same or substantially similar function in the system 100.

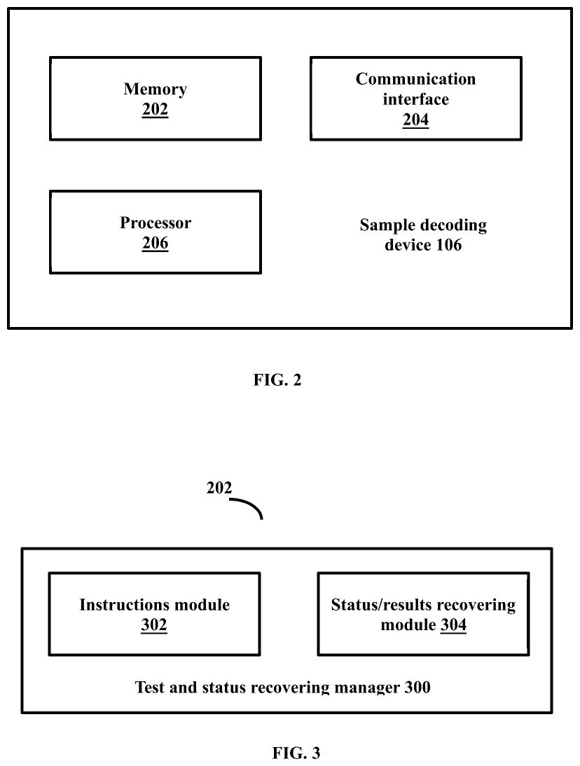

FIG. 2 is an example block diagram depicting various components of the sample decoding device 106, according to embodiments as disclosed herein. The sample decoding device 106 includes a memory 202, a communication interface 204, and a controller/processor 206.

The memory 202 may store at least one of, but is not limited to, request (including the name of the test, the size of the test, the number of positives estimated, or the like) received from the users, the results/status of the biological samples associated with each test, the viral load of each biological sample, and so on. The memory 202 may also store a test manager 300, which may be executed on the processor 206 for determining the instructions related to the pooling of the biological samples and recovering the status of each biological sample. The memory 202 referred herein may include at least one type of storage medium, from among a flash memory type storage medium, a hard disk type storage medium, a multi-media card micro type storage medium, a card type memory (for example, an SD or an XD memory), random-access memory (RAM), static RAM (SRAM), read-only memory (ROM), electrically erasable programmable ROM (EEPROM), programmable ROM (PROM), a magnetic memory, a magnetic disk, or an optical disk.

The communication interface 204 may be configured to enable the sample decoding device 106 to communicate with at least one of the user devices, or the like through the communication network.

The processor 206 may include at least one of, a single processer, a plurality of processors, multiple homogeneous or heterogeneous cores, multiple CPUs of different kinds, a microcontroller, and other accelerators. The processor may be configured to execute the test manager 300 to determine the instructions for pooling of the biological samples based on the requested size of test by the user and to recover the status of each biological sample based on the received input cycle threshold values of each pool of biological samples from the user.

As depicted in FIG. 3, the test and status recovering manager 300 includes an instructions module 302, and a status/results recovering module 304. The instructions module 302 may be configured to provide the instructions to the user for pooling and testing of the biological samples. The instructions module 302 may receive the request (including the including the name of the test, the size of the test, the number of positives estimated, or the like) from the user for the instructions related to the pooling and testing of the biological samples. The instructions module 302 may determine the pooling matrix for the received request from the user. In an embodiment, the instructions module 302 may determine the pooling matrix by creating the function that may be the dot product of the pair of columns and optimizing the function in the gradient descent method and simulated annealing method. In another embodiment, the instructions module 302 may determine the pooling matrix using the Steiner triples system. The created pooling matrix may be the adjacency matrix with the sparse expanders. The pooling matrix may include the entries of 0's and 1's. The rows of the pooling matrix indicate the pools to be created for testing of the biological samples, the columns of the pooling matrix indicate the biological samples to be tested, and the entries of 1's in each column indicate the pools for including each biological sample corresponding to each column. The instructions module 302 provides the instructions including the number of pools/tests to be created for the biological samples (that have to be tested and the two or different pools for each biological sample to the user in response to the received request. According to the received instructions, the user may pool the biological samples and perform the RT-qPCR test on each pool to determine the Ct values corresponding to each pool.

The status recovering module 304 may be configured to recover the status of each biological sample. The status recovering module 304 uses the COMP, the binary non-adaptive method and determines the pools that are negative and the pools that are positive from the Ct values of each pool. The status recovering module 304 determines that the pools are negative (i.e., the biological samples of the corresponding pools do not include the viruses), if the Ct values have not been obtained for the pools. The status recovering module 304 determines that the pools are positive (i.e., the one or more biological samples included in the corresponding pools include the viruses), if the Ct values have been obtained for the pools. The status recovering module 304 converts the Ct values of the pools that have been identified as positive to the viral load. The status recovering module 304 uses the compressed sensing method to construct the noisy linear equation by considering the converted quantitative measure of the viral load of the pools and the pooling matrix created for the testing of the biological samples.

The status recovering module 304 solves the noisy linear equation to derive the status of each biological sample. The status recovering module 304 communicates the derived status of each biological sample to the user.

FIGS. 2 and 3 show exemplary blocks of the sample decoding device 106, but it is to be understood that other embodiments are not limited thereon. In other embodiments, the sample decoding device 106 may include less or a greater number of blocks. Further, the labels or names of the blocks are used only for illustrative purpose and does not limit the scope of the embodiments herein. One or more blocks can be combined together to perform the same or substantially similar function in the sample decoding device 106.

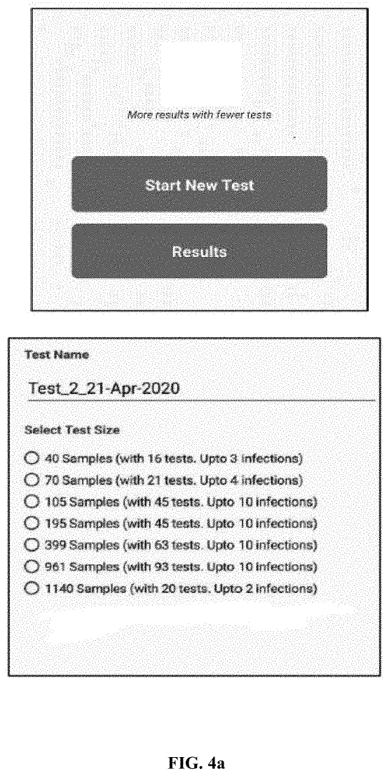

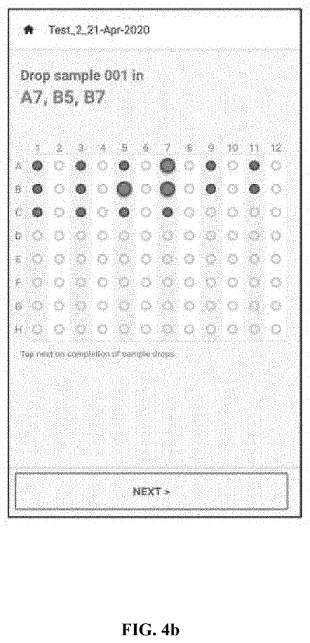

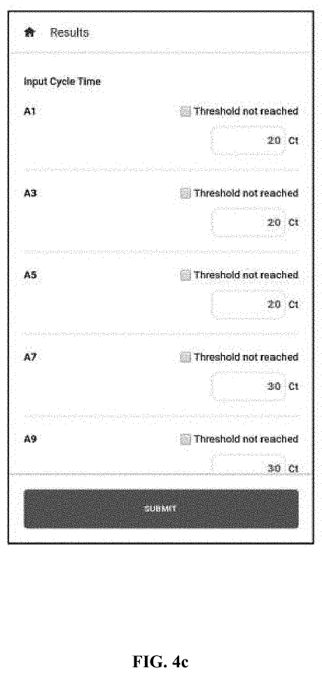

FIGS. 4a, 4b and 4c are example conceptual diagrams depicting determination of the viruses in the biological samples of the individuals using the single round pooling method, according to embodiments as disclosed herein.

The sample decoding device 106 receives a request for the instructions for performing the pooling and tests on the biological samples of the individuals, from the user (through the application configured on the user device 102). The tests may be RT-qPCR tests. The request may include a unique name for the test (with date and time), a size of the test, or the like. The size of the test may indicate a number of biological samples to be tested and a number of positives expected (as depicted in FIG. 4a).

The sample decoding device 106 creates/constructs the pooling matrix for the pooling and testing of the biological samples, based on the requested size of the test. The sample decoding device 106 creates the matrix using Steiner triples system. The matrix indicates the number of pools for the requested size of the test and the two or more pools (for example: 3 different pools) in which each biological sample has to be pipetted/combined. The sample decoding device 106 may provide the instructions to the user in response to the received request from the user. The instructions may be related to the number of pools to be tested for the requested number of biological samples and the three pools for each biological sample (as depicted in FIG. 4b). For example, as depicted in FIG. 4b, grey dots indicate the pools/wells in which a first sample (sample 001) has to be combined/pipetted.

On receiving the instructions from the sample decoding device 106, the user may select the PCR reaction plate with the wells, according to the instructions received from the sample decoding device 106. The number of wells may correspond to the number of pools to be tested (which has been indicated in the instructions). In an example herein, consider that the user has selected 0.2 milliliter (mL) 96 well PCR reaction plate. The user may place the selected PCR reaction plate on a well cold block/ice, number the biological samples (for example; sample 001, sample 002, or the like) and prepare to pool each of the biological samples into the three appropriate wells.

The user pipettes each biological sample into the three appropriate wells/pools, according to the three different pools indicated for the corresponding biological sample in the instructions received from the sample decoding device 106. The user may pipette any volume of the biological samples into the appropriate pools/wells of the PCR reaction plate. In an example, the user may pipette between 2 μL and 10 μL of the biological sample into the appropriate pool.

In an embodiment, the user may extract the RNA from the biological samples to be tested and then pipette each extracted RNA corresponding to each biological sample into the three wells/pools of the PCR reaction plate, according to the three different pools indicated for the corresponding biological sample in the instructions received from the sample decoding device 106. The user may extract the RNA using suitable RNA extraction kits and concentrate the extracted RNA using a speed-vac to increase sensitivity of the test. In another embodiment, the user may pipette each biological sample into the three wells/pools of the PCR reaction plate, according to the three different pools indicated for the corresponding biological sample in the instructions received from the sample decoding device 106 and then extract the RNA from the biological samples of each pool.

On completing the pipetting/pooling of all the biological sampled into the appropriate wells of the PCR reaction plate, the user may tightly seal the PCR reaction plate to prevent loss of the biological samples and spin down the PCR reaction plate (for example: for 30 seconds) to collect all liquid at the bottom. The PCR reaction plate then may be gently vortexed by the user (for example: for 5 seconds) to ensure that the biological samples in each well is uniformly mixed and the PCR reaction plate may then again spin down (for example: for 30 seconds). The user then performs the

RT-qPCR tests on the biological samples of each pool using the PCR machine. The RT-qPCR tests may be performed according to the instructions of the Real-Time RT-PCR diagnostic panel. In an example, in a typical PCR reaction, 5 ul of the pooled samples may be used in a 25 ul RT-PCR reaction. The PCR machine provides the Ct values data corresponding to each pool to the user, on completion of performing the RT-PCR tests on all the pools. The user may communicate Ct values data of each pool to the sample decoding device 106, as depicted in FIG. 4c. The Ct values data of each pool may indicate that Ct values have not reached for the pool or the Ct values.

Based on the received Ct values data of each pool, the sample decoding device 106 determines the pools that are positive and the pools that are negative using the binary non-adaptive method. The sample decoding device 106 converts the Ct values of the pools that have been identified as positive to the viral load. The sample decoding device 106 uses the compressed sensing method to construct the noisy linear equation by considering the converted quantitative measure of the viral load of the pools and each biological sample and the pooling matrix created for the testing of the biological samples. The sample decoding device 106 solves the noisy linear equation for each biological sample to determine the associated viral load. Thus, deriving the status of each biological sample. The sample decoding device 106 communicates the derived status of each biological sample to the user. The status of each biological sample indicates whether the viruses are present in the biological sample or not.

Thus, status of the plurality of biological samples may be recovered in the single round of testing.

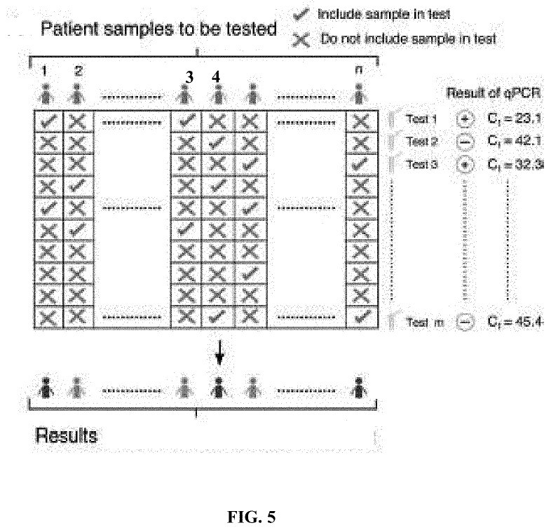

FIG. 5 is an example diagram depicting recovering the status of the plurality of biological samples using the quantitative, non-adaptive and single round pooling method, according to embodiments as disclosed herein.

The sample decoding device 106 receives the request for instructions related to pooling and testing of the biological samples from the user through the user device 102. The request includes the size of the test indicating the number of biological samples to be tested and a number of positives estimated. The sample decoding device 106 constructs the matrix for the requested size of test, based on the Steiner triples system. The matrix may be the sparse matrix indicating a number of pools for the requested size of the test and the different two or more pools for each biological sample. In an embodiment, the sample decoding device 106 determines three different pools for each biological sample. The sample decoding device 106 provides the instructions to the user indicating the number of pools for the requested size of the test and the three different pools for each biological sample.

On receiving the instructions from the sample decoding device 106, the user may perform the pooling of the biological samples by pipetting each biological sample into the indicated three of the different pools/wells of the PCR reaction plate. The user may perform the RT-qPCR test on each pool and determines the Ct values of each pool. The RT-qPCR may include the qPCR amplification performed on the RNA fragments/templates or the qPCR amplification performed on the DNA fragments/templates of the biological samples of each pool. The user may communicate the Ct values of each pool to the sample decoding device 106, on completing the RT-qPCR tests on all the pools of biological samples.

The sample decoding device 106 converts the Ct values of each pool into the quantitative measure of the viral load. The sample decoding device 106 uses the compressed sensing method to construct the noisy linear equation based on the quantitative measure of the viral load and solves the noisy linear equation to recover the status of each biological sample.

Consider an example scenario, as depicted in FIG. 4, ‘n’ number of biological samples of patients have been combined/pipetted into ‘m’ number of pools. The RT-qPCR test is performed on each of the ‘m’ number of pools and the Ct values of each pool is determined. Thereafter, the status of each of the ‘n’ biological samples is determined in the single round by converting the Ct values of each pool into the viral load. In an example herein, the status of the 1^{st}, 4^{th} and m^{th} biological samples may be determined as positive, since the one or more pools/tests associated with the 1st, 4^{th} and m^{th} biological samples are positive. The status of the other biological samples may be determined as negative, since the one or more pools/tests associated with the corresponding biological samples are negative.

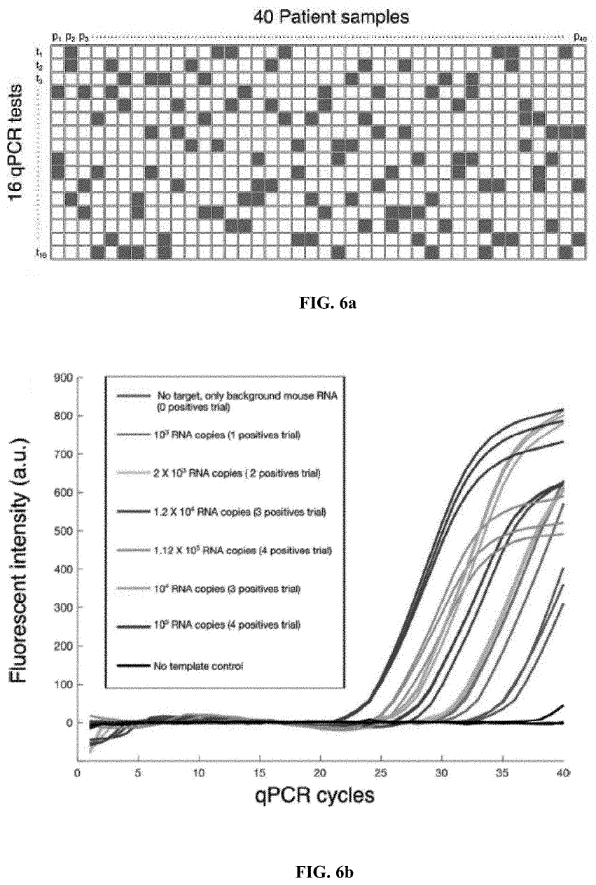

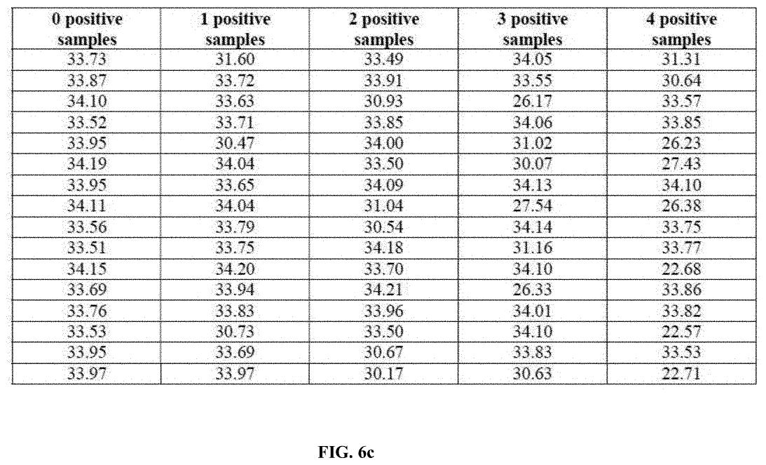

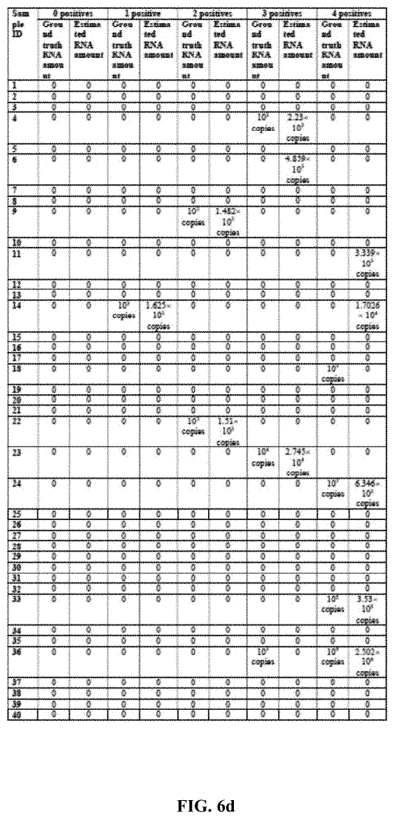

FIGS. 6a. 6b, .6c, and 6d are example diagrams depicting testing of the biological samples of 40 patients using the quantitative, non-adaptive and single round pooling method, according to embodiments as disclosed herein.

Consider an example scenario, wherein the sample decoding device 106 receives the request from the user for the instructions related to pooling and testing of the biological samples, wherein the request indicates that 40 biological samples have to be tested with a number of positives estimated. In such a scenario, the sample decoding device 106 creates a 16×40 matrix, which indicates that 40 biological samples may be pooled into 16 pools (as depicted in FIG. 6a). Thus, 40 biological samples may be tested in 16 pools. The 16×40 matrix (as depicted in FIG. 6a) directs that each sample be distributed, and hence tested, two or three times while the number of samples per pool vary from six to nine, with a median pool size of seven. In an example, the 16x40 matrix created with respect to zero positives estimated, one positive (103 copies) estimated, two positives (103) estimated, three positives estimated, and four positives estimated. The sample decoding device 106 communicates the instructions to the user, indicating the 16 pools for the 40 biological samples and three different pools for each of the 40 biological samples.

On receiving the instructions, the user pipettes each of the 40 biological samples into the indicated three pools of the 16 pools/wells of the PCR reaction plate. The user performs the RT-qPCR test on each pool of biological samples and determines the input cycle threshold value for each pool of biological samples. The RT-qPCR may include the qPCR amplification performed on the RNA fragments/templates or the qPCR amplification performed on the DNA fragments/templates of the biological samples of each pool Amplification curves obtained by performing the RT-qPCR test on each of 16 pools of biological samples are depicted in FIG. 6b. The user may use the amplification curves to derive the input cycle threshold value for each pool. The user may communicate the input cycle threshold value of each pool of the 16 pools to the sample decoding device 106, which recovers the status of each of the 40 biological samples based on the input cycle threshold value of each pool of biological samples.

In an example, the input cycle threshold values determined for each of the 16 pools corresponding to five different trials (0, 1, 2, 3 or 4 positive samples out of the total of 40 biological samples) is depicted in an example table of FIG. 6c. Further, as depicted in FIG. 6d, ground truth RNA amounts for each of the 40 samples have been compared to the RNA amounts estimated using the single round based pooling method.

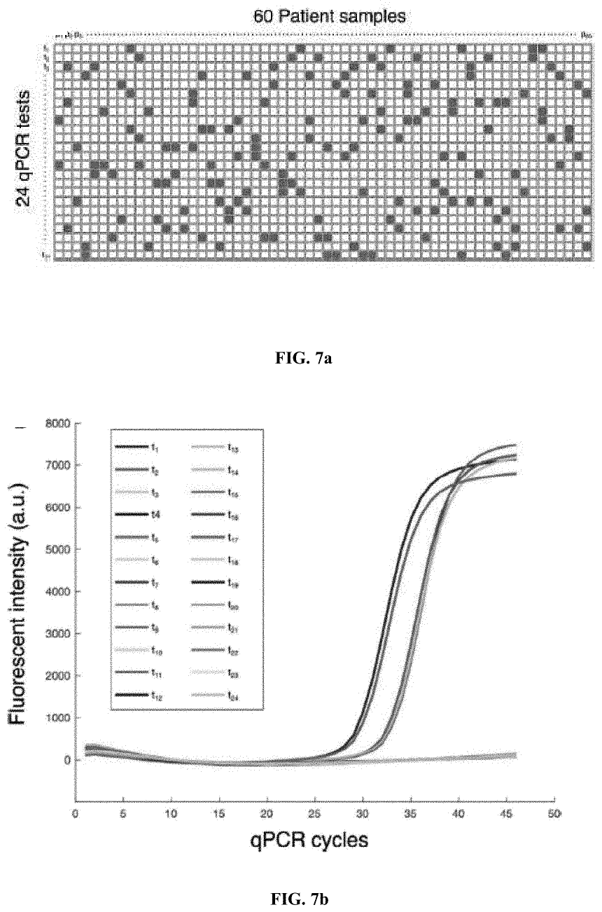

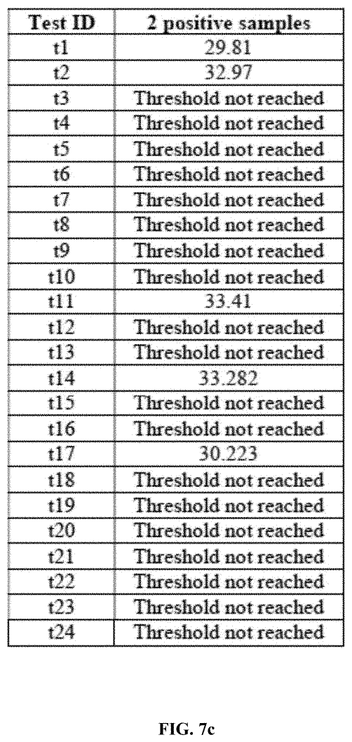

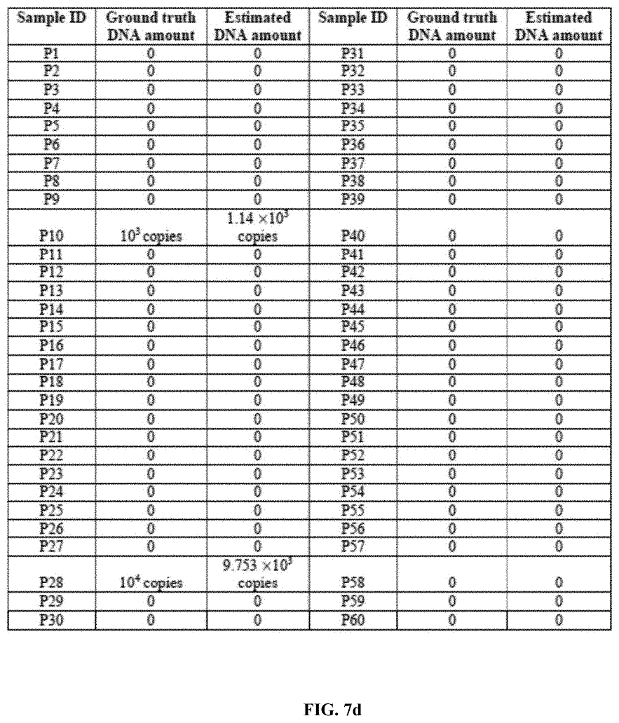

FIGS. 7a. 7b, 7c, and 7d are example diagrams depicting testing of the biological samples of 60 patients using the quantitative, non-adaptive and single round pooling method, according to embodiments as disclosed herein.

Consider an example scenario, wherein the sample decoding device 106 receives the request from the user for the instructions to test the biological samples, wherein the request indicates that 60 biological samples have to be tested with a number of positives estimated. In such a scenario, the sample decoding device 106 creates a 24×60 matrix, which indicates that 60 biological samples may be pooled into 24 pools (as depicted in FIG. 5a). Thus, 60 biological samples may be tested in 24 pools. The 24 ×60 matrix (as depicted in FIG. 7a) directs that each sample be distributed, and hence tested, two or three times while the number of samples per pool vary from six to nine, with a median pool size of seven. In an example, the 24×60 matrix created with respect to zero positives estimated, one positive copies estimated, two positives estimated, three positives estimated, and four positives estimated. The sample decoding device 106 communicates the instructions to the user, indicating the 24 pools for the 60 biological samples and three different pools for each of the 60 biological samples.

On receiving the instructions, the user pipettes each of the 60 biological samples into the indicated three pools of the 24 pools/wells of the PCR reaction plate. The user performs the RT-qPCR test on each pool of biological samples and determines the input cycle threshold value for each pool of biological samples. The RT-qPCR may include the qPCR amplification performed on the RNA fragments/templates or the qPCR amplification performed on the DNA fragments/templates of the biological samples of each pool Amplification curves obtained by performing the RT-qPCR test on each of 24 pools of biological samples is depicted in FIG. 7b. The user may use the amplification curves to derive the input cycle threshold value for each pool. The user may communicate the input cycle threshold value of each pool of the 24 pools to the sample decoding device 106, which recovers the status of each of the 60 biological samples based on the input cycle threshold value of each pool of biological samples.

In an example, the input cycle threshold values determined for each of the 24 pools corresponding to two positive samples out of the total of 60 biological samples is depicted in an example table of FIG. 7c. Further, as depicted in FIG. 7d, ground truth RNA amounts for each of the 60 samples have been compared to the RNA amounts estimated using the single round based pooling method.

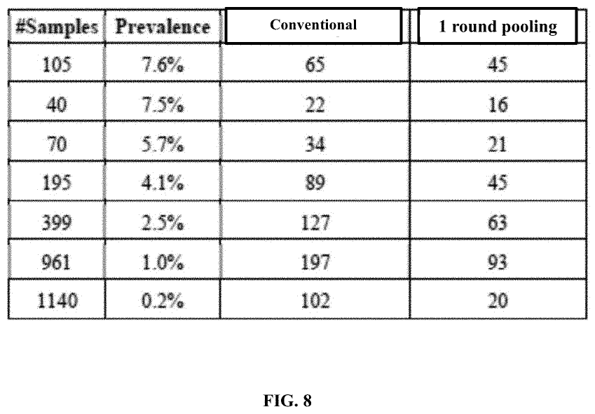

FIG. 8 is an example table depicting an improved throughput in recovering the status of the biological samples using the quantitative, the non-adaptive and single round pooling method compared to a conventional approach, according to embodiments as disclosed herein.

In a conventional approach, a two round poling may be used to recover the status of each biological sample. In contrast, embodiments herein may recover the status of plurality of biological samples in the single round using the quantitative, non-adaptive pooling method. Thereby, increasing the throughout by recovering the results/status in one round and reducing the required number of tests on the biological samples.

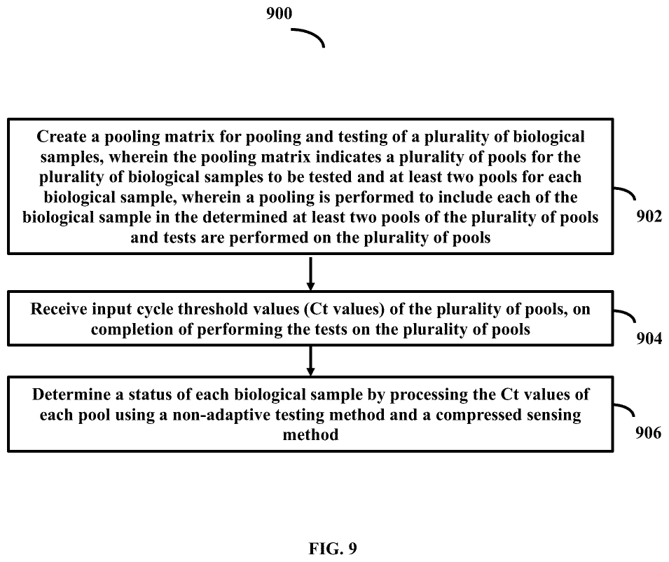

FIG. 9 is a flow diagram depicting a method for determining the viruses in the biological samples of the individuals, according to embodiments as disclosed herein.

At step 902, the method includes creating, by the sample decoding device 106, the pooling matrix for pooling and testing of the plurality of biological samples. The pooling matrix indicates the plurality of pools for the plurality of biological samples to be tested and the at least two pools for each biological sample. In an embodiment, the sample decoding device 106 may create the pooling matrix by creating the function that is the dot product of pair of columns and optimizing the function in the gradient descent method and simulated annealing method. In another embodiment, the sample decoding device 106 may create the pooling matrix using the Steiner triples system. On creating the pooling matrix, the pooling is performed to include each of the biological sample in the determined at least two pools of the plurality of pools and tests are performed on the plurality of pools.

At step 904, the method includes receiving, by the sample decoding device 106, the Ct values of the plurality of pools, on completion of performing the tests on the plurality of pools.

At step 906, the method includes determining, by the sample decoding device 106, the status of each biological sample by processing the Ct values of each pool using the non-adaptive testing method and the compressed sensing method, wherein the status of each biological sample indicates whether the viruses are present in the biological samples or not. The various actions, acts, blocks, steps, or the like in the method and the flow diagram 900 may be performed in the order presented, in a different order or simultaneously. Further, in some embodiments, some of the actions, acts, blocks, steps, or the like may be omitted, added, modified, skipped, or the like without departing from the scope of the invention.

Embodiments herein test each biological sample a number of times (for example; thrice) as part of different pools and recover results/status of each biological sample using input cycle threshold values of each pool of biological samples. Testing of each biological sample and recovering the results of each biological sample may be performed using:
Sparse Pooling Matrix: The sparse pooling matrix may be created for pooling the biological samples, based on combinatorial designs known as Steiner triples. Each biological sample may be combined into 3 pools, as opposed to 6 pools in conventional approaches, which reduces pipetting time by half.
Noise model: The explicit noise model may be constructed and resolved to recover the status of each biological sample, where the explicit noise model may be constructed by considering that the input cycle threshold values of each pool are measured with additive Gaussian noise, thereby leading to a multiplicative noise on a reconstruction vector.
Sparsity estimator and graceful failure: A status of each biological sample may be recovered by maintaining very high sensitivity of the test and determining a list of “suspected positives” and an estimate of how many of these suspected positives are truly positives. Thus, increasing performance and throughput with reduced testing rounds.

The embodiments disclosed herein can be implemented through at least one software program running on at least one hardware device and performing network management functions to control the network elements. The network elements shown in FIGS. 1, 2, and 3 include blocks which can be at least one of a hardware device, or a combination of hardware device and software module.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the spirit and scope of the embodiments as described herein.

I/We claim:

1. A method for determining viruses in biological samples, the method comprising:
creating, by a sample decoding device (106), a pooling matrix for pooling and testing of a plurality of biological samples, wherein the pooling matrix indicates a plurality of pools for the plurality of biological samples to be tested and at least two pools for each biological sample, wherein a pooling is performed to include each of the biological sample in the determined at least two pools of the plurality of pools and tests are performed on the plurality of pools;
receiving, by the sample decoding device (106), input cycle threshold values (Ct values) of the plurality of pools, on completion of performing the tests on the plurality of pools; and
determining, by the sample decoding device (106), a status of each biological sample by processing the Ct values of each pool using a non-adaptive testing method and a compressed sensing method, wherein the status of each biological sample indicates whether the viruses are present in the biological samples or not.

2. The method of claim 1, wherein the viruses are single-stranded Ribonucleic acid (RNA) viruses including at least one of, Coronavirus disease of 2019 (COVID-19), and Severe Acute Respiratory Syndrome (SARS).

3. The method of claim 1, wherein creating the pooling matrix includes:
receiving a request from a user for the pooling and testing of the plurality of biological samples, wherein the request includes information about at least one of, a name of a test, and a size of the test, wherein the size of the test indicates a total number of biological samples to be tested and a number of biological samples estimated as positive out of the total number of biological samples;
creating the pooling matrix for the requested size of the test.

4. The method of claim 3, wherein the pooling matrix is an adjacency matrix with sparse expanders, wherein the pooling matrix includes:
a plurality of rows indicating the plurality of pools to be created for testing of the plurality of biological samples;
a plurality of columns indicating the plurality of biological samples to be tested; and
at least two entries of 1's in each column indicating the at least two pools for including each biological sample corresponding to each column.

5. The method of claim 3, wherein creating the pooling matrix includes:
creating a function that is a dot product of the plurality of columns; and
optimizing the created function in a gradient descent method and a simulated annealing method.

6. The method of claim 3, wherein the pooling matrix is created using a Steiner triples system.

7. The method of claim 1, wherein performing the pooling and testing of the plurality of biological samples includes:
selecting a Polymerase Chain Reaction (PCR) reaction plate with a plurality of wells based on the created pooling matrix, wherein the plurality of wells corresponds to the plurality of pools determined for the plurality of biological samples to be tested;
combining each biological sample into at least two wells of the PCR reaction plate, wherein the at two wells corresponds to the at least two pools determined for each biological sample; and
performing tests on the combined biological samples of the plurality of pools to determine the Ct values of the plurality of pools, wherein the tests include quantitative reverse transcription Polymerase Chain Reaction (PCR) (RT-qPCR) tests.

8. The method of claim 1, wherein determining, by the sample decoding device (106), the status of each biological sample includes:
identifying pools from the plurality of pools that are negative and pools from the plurality of pools that are positive by analyzing the Ct values of the plurality of pools using a binary non-adaptive testing method, wherein the binary non-adaptive testing method includes a Combinatorial Orthogonal Matching Pursuit (COMP) method;
converting the Ct values of the pools that are identified as positive into quantitative measures of viral loads;
performing a compressed sensing method to construct a noisy linear equation by considering the quantitative measures of the viral loads of the pools, the pooling matrix and a viral load of each biological sample; and
solving the constructed noisy linear to determine the viral load of each biological sample, wherein the determined viral load of each biological sample indicates the status of each biological sample.

9. The method of claim 8, wherein the pools identified as negative correspond to pools for which the Ct values are not determined, and the pools identified as positive correspond to pools for which the Ct values are determined.

10. The method of claim 8, wherein the noisy linear equation is solved using at least one of, a Non-negative least absolute shrinkage and selection operator (NN-LASSO) method, a Non-negative Orthogonal Matching Pursuit (NNOMP) method, a Sparse Bayesian Learning (SBL) method, and a brute force search method.

11. A sample decoding device (106) comprising:
a memory (202); and
a processor (206) coupled to the memory (202) configured to:
create a pooling matrix for pooling and testing of a plurality of biological samples, wherein the pooling matrix indicates a plurality of pools for the plurality of biological samples to be tested and at least two pools for each biological sample, wherein a pooling is performed to include each of the biological sample in the determined at least two pools of the plurality of pools and tests are performed on the plurality of pools;
receive input cycle threshold values (Ct values) of the plurality of pools, on completion of performing the tests on the plurality of pools; and
determine a status of each biological sample by processing the Ct values of each pool using a non-adaptive testing method and a compressed sensing method, wherein the status of each biological sample indicates whether the viruses are present in the biological samples or not.

12. The sample decoding device (106) of claim 11, wherein the viruses are single-stranded Ribonucleic acid (RNA) viruses including at least one of, Coronavirus disease of 2019 (COVID-19), and Severe Acute Respiratory Syndrome (SARS).

13. The sample decoding device (106) of claim 11, wherein the processor (206) is further configured to:
receive a request from a user for the pooling and testing of the plurality of biological samples, wherein the request includes information about at least one of, a name of a test, and a size of the test, wherein the size of the test indicates a total number of biological samples to be tested and a number of biological samples estimated as positive out of the total number of biological samples; and
create the pooling matrix for the requested size of the test.

14. The sample decoding device (106) of claim 13, wherein the pooling matrix is an adjacency matrix with sparse expanders, wherein the pooling matrix includes:
a plurality of rows indicating the plurality of pools to be created for testing of the plurality of biological samples;
a plurality of columns indicating the plurality of biological samples to be tested; and
at least two entries of 1's in each column indicating the at least two pools for including each biological sample corresponding to each column.

15. The sample decoding device (106) of claim 13, wherein the processor (206) is further configured to:
create a function that is a dot product of the plurality of columns; and
optimize the created function in a gradient descent method and a simulated annealing method, wherein the created and optimized function is the pooling matrix.

16. The sample decoding device (106) of claim 13, wherein the pooling matrix is created using a Steiner triples system.

17. The sample decoding device (106) of claim 11, wherein performing the pooling and testing of the plurality of biological samples includes:
selecting a Polymerase Chain Reaction (PCR) reaction plate with a plurality of wells based on the created pooling matrix, wherein the plurality of wells corresponds to the plurality of pools determined for the plurality of biological samples to be tested;
combining each biological sample into at least two wells of the PCR reaction plate, wherein the at two wells corresponds to the at least two pools determined for each biological sample; and
performing tests on the combined biological samples of the plurality of pools to determine the Ct values of the plurality of pools, wherein the tests include quantitative reverse transcription Polymerase Chain Reaction (PCR) (RT-qPCR) tests.

18. The sample decoding device (106) of claim 13, wherein the processor (206) is further configured to:
identify pools from the plurality of pools that are negative and pools from the plurality of pools that are positive by analyzing the Ct values of the plurality of pools using the non-adaptive testing method, wherein the binary non-adaptive testing method includes a Combinatorial Orthogonal Matching Pursuit (COMP) method;
convert the Ct values of the pools that are identified as positive into quantitative measures of viral loads;
perform the compressed sensing method to construct a noisy linear equation by considering the quantitative measures of the viral loads of the pools, the pooling matrix and a viral load of each biological sample; and
solve the constructed noisy linear to determine the viral load of each biological sample, wherein the determined viral load of each biological sample indicates the status of each biological sample.

19. The sample decoding device (106) of claim 18, wherein the pools identified as negative correspond to pools for which the Ct values are not determined, and the pools identified as positive correspond to pools for which the Ct values are determined.

20. The sample decoding device (106) of claim 18, wherein the noisy linear equation is solved using at least one of, a Non-negative least absolute shrinkage and selection operator (NN-LASSO) method, a Non-negative Orthogonal Matching Pursuit (NNOMP) method, a Sparse Bayesian Learning (SBL) method, and a brute force search method.
